(19) United States
(12) Patent Application Publication
(10) Pub. No.: US 2005/0187497 A1
(43) Pub. Date: Aug. 25, 2005

(54) Electrical impulse apparatus for facial massage

(76) Inventor: Thi Ngoc Phuong Nguyen, Sales Le Chateau (FR)

Correspondence Address: Yi-Wen Tseng, 4331 Stevens Battle Lane, Fairfax, VA, 22033, US

(21) Appl. No.: 10/784,248

(22) Filed: Feb. 24, 2004

(51) Int. Cl. A61N 1/18

(52) U.S. Cl. 601/21; 601/15

(57) ABSTRACT

An electrical impulse apparatus for facial massage includes a microprocessor, an impulse generator, and a pad. The microprocessor controls the impulse generator to generate pulsed signals to two electrodes on the pad via two conductive lines, respectively. When the pad is attached to the facial skin, by using a set of switch buttons to power on/off the pulse generation and then in turn to increase/decrease the strength of the pulse generation, the electro-muscle stimulation (EMS) is applied to facial muscles and thereby increase the blood circulation in the surrounding area. As such, the muscle tone and electricity of facial tissue are enhanced to minimize the wrinkle and sagging of facial muscles due to aging process.

### BACKGROUND OF THE INVENTION

The present invention relates to an electrical impulse apparatus for facial massage, and more particular, to a facial toning apparatus which provides electro-muscle stimulation to exercise facial muscles.

It is well known of that as a person ages the facial muscles of an individual lose tone and elasticity and tend to sag. The loss of tone and elasticity of the facial muscles creates wrinkles and jowls on a person's face. No one, especially woman, wants age lines or depressions which become more or less prominent parts of his or her facial features and are generally considered undesirable in appearance. As such, many products and devices exist out in the market that claim to slow, stop or reverse the aging process of the skin. One of them is by clinic therapy, such as by face-lift or botulinus treatment. Particularly common are the topical creams for moisturizing, naturalizing and contracting face or body skin. Various creams, lotions and skin conditioners are available which are purported to reduce or eliminate the negative aspects of the appearance of these age lines. However, invariably, they must to be applied continually and there is certainly no immediate effect.

### BRIEF SUMMARY OF THE INVENTION

The present invention provides an electrical impulse apparatus for facial massage which generates impulse to the epidermis of the skin to increase the blood circulation in the surrounding area and stimulate the contraction of the muscle. Therefore, the tone and elasticity of the facial skin can be remained so as to minimize the wrinkle and sagging of facial muscles and delay the aging of the skin.

Furthermore, the present invention provides an electrical impulse apparatus for facial massage which utilizes a pad attachable on face to transmit electro-muscle stimulation (EMS) without penetration treatment to human body. Therefore, there is no invasion of the tissue and more safety is obtained.

Still further, the present invention provides an electrical impulse apparatus for facial massage which is cost-effective by using electrical power to maintain the application. Therefore, it is affordable for user.

The electrical impulse apparatus for facial massage provided by the present invention includes a circuit to generate pulsed signals and then the signals are transmitted to two electrodes mounted on the pad by conductive lines. Therefore, when the pad is attached to the facial skin, EMS is applied to exercise facial muscles to improve muscle tone and strength and to against aging process.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

These, as well as other features of the present invention, will become apparent upon reference to the drawings wherein:

FIG. 1 shows a perspective view of a first preferred embodiment provided by the present invention;

FIG. 2 shows a perspective view of a second preferred embodiment provided by the present invention;

FIG. 3 shows a circuit diagram of the present invention;

FIG. 4 shows a flowchart for operating the present invention;

FIG. 5 shows an operation cycle of the present invention; and

FIG. 6 shows an application to attach on the face.

### DETAILED DESCRIPTION OF THE INVENTION

Reference will now be made in detail to the preferred embodiments of the present invention, examples of which are illustrated in the accompanying drawings. Wherever possible, the same reference numbers are used in the drawings and the description to refer to the same or like parts.

Body exercise is triggered by nervous system. Muscles provide strength to lead skeleton to complete. The nervous system includes central and peripheral parts. The nerve fibers of the peripheral nervous system execute the response of the central due to the stimuli. The exercise message sent by brain is an impulse which is delivered from neurons one by one to muscle. This impulse reflects a voltage change. That is, a voltage value of the neuron is −70 mV at static, and +30 mV at dynamic state. Thus, the impulse has a waveform as a pulse.

One the other hand, electro-muscle stimulation (EMS) is well known in the medical art. This technology utilized a conductive pad or electrode to externally apply a very weak current to a muscle or group of muscles and thereby cause them to contract. The electrode receives an electric stimulation signal from an external voltage/current source, such as an EMS machine. The stimulation signal can be adjusted in amplitude, polarity, frequency, waveform, etc. EMS is useful for the general exercise of functional muscles to improve muscle tone and strength.

Furthermore, the facial condition represents the first impression of the age. Dry skin, loss of firmness and elasticity, wrinkle and sagging accompany the aging process. If these negative aspects of the appearance is improved, an anti-aging effect is obtained. Therefore, this invention introduces impulses of EMS to facial skin so as to exercise the muscles to achieve the goal.

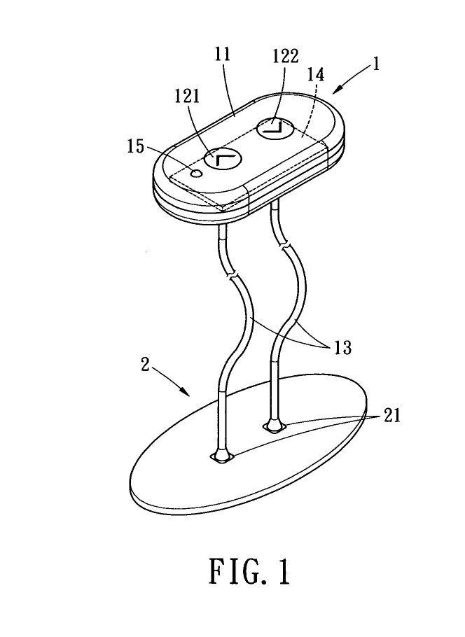

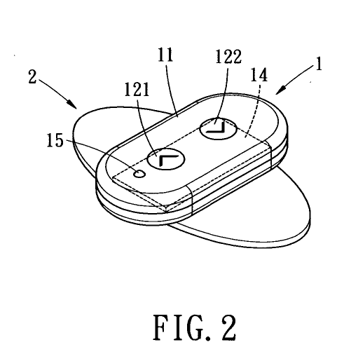

As shown in FIG. 1, the electrical impulse apparatus for facial massage provided by the present invention includes a controller 1 and a pad 2. The controller 1 includes a circuit board 14 installed inside a housing 11. At least two buttons 121, 122 are set on the surface of the housing 11. One of the buttons 121 is served to power on the apparatus when the apparatus is power-off, and in turn to increase the strength of the output impulse when the apparatus is power-on. Similarly, the other button 122 is to decrease the strength and power off the apparatus. It can utilize different kind of pushing arrangement to perform this function, such as when the apparatus is power-on, pressing the button 122 will continuously reduce the strength of the output impulse until the power is off. Moreover, a light emitting device 15 is installed on the housing 11 to indicate the power of the apparatus is turned on or off. The circuit board 14 includes two conductive lines 13 connected to two electrodes 21, respectively. The electrodes 21 are mounted on the pad 2 with a fixed arrangement. The pad 2 has one surface attachable to the facial skin. The controller 1 and the pad 2 can be separate by the connection of the conductive lines 13. As such, user can hold the controller 1 in hand to perform facial massage. However, controller 1 and the pad 1 can be combined together as shown in FIG. 2. In this preferred embodiment, there is no need of the external conductive lines 13.

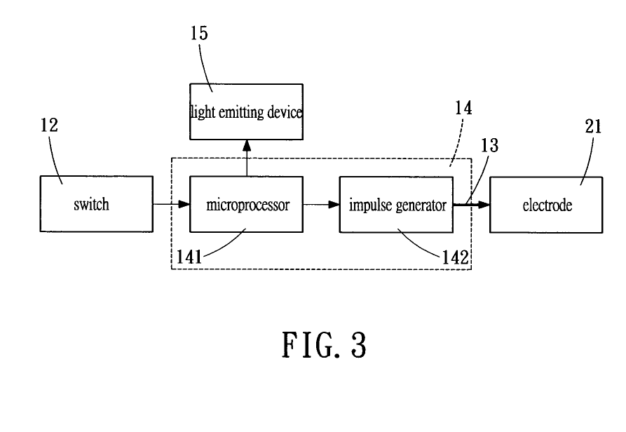

FIG. 3 shows the circuit diagram of the electrical impulse apparatus. As shown, the circuit board 14 includes a microprocessor 141 and an impulse generator 142. The impulse generator 142 is controlled by the microprocessor 141 to generate pulsed signals. The signals may transmit to the electrodes 21 via the external conductive lines 13. The switch of buttons 12 and the light emitting device 15 are connected to the microprocessor 141. User pushes the switch 12 to operate the microprocessor 141 to output the impulse. The microprocessor 141 also controls the light emitting device 15 to indicate the on/off status of the apparatus.

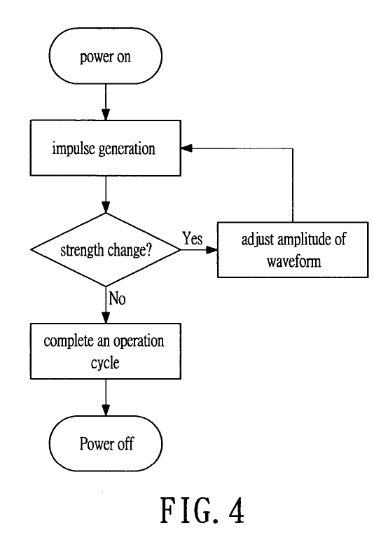

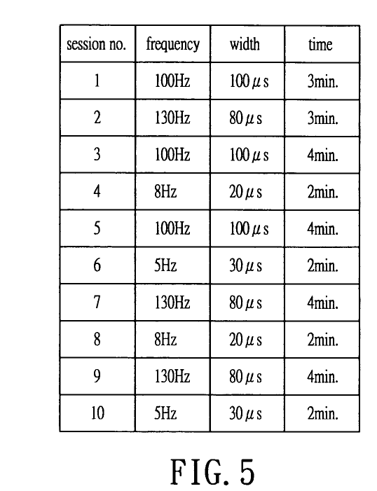

The flowchart for operating the electrical impulse apparatus is illustrated in FIG. 4. As shown, after the apparatus is powered on, the pulsed signals are sequentially sending from the impulse generator 142 to the electrodes 21. If the strength of the output impulse is changed by user, the impulse generator is controlled by the microprocessor 141 to adjust the amplitude of the pulse wave with respect to the change. After an operation cycle is finished, the microprocessor 141 will automatically power off the apparatus until it is activated by user again. The operation cycle is shown in FIG. 5. There is a totally continuous thirty-minute cycle including ten sessions. The first session outputs the impulse with the pulse wave 100 Hz in frequency and 100 μs in width for continuous three minutes. The second session outputs the impulse with the pulse wave 130 Hz in frequency and 80 μs in width for continuous three minutes. The third session outputs the impulse with the pulse wave 100 Hz in frequency and 100 μs in width for continuous four minutes. The fourth session outputs the impulse with the pulse wave 8 Hz in frequency and 20 μs in width for continuous two minutes. The fifth session outputs the impulse with the pulse wave 100 Hz in frequency and 100 μs in width for continuous four minutes. The sixth session outputs the impulse with the pulse wave 5 Hz in frequency and 30 μs in width for continuous two minutes. The seventh session outputs the impulse with the pulse wave 130 Hz in frequency and 80 μs in width for continuous four minutes. The eighth session outputs the impulse with the pulse wave 8 Hz in frequency and 20 μs in width for continuous two minutes. The ninth session outputs the impulse with the pulse wave 130 Hz in frequency and 80 μs in width for continuous four minutes. The tenth session outputs the impulse with the pulse wave 5 Hz in frequency and 30 μs in width for continuous two minutes.

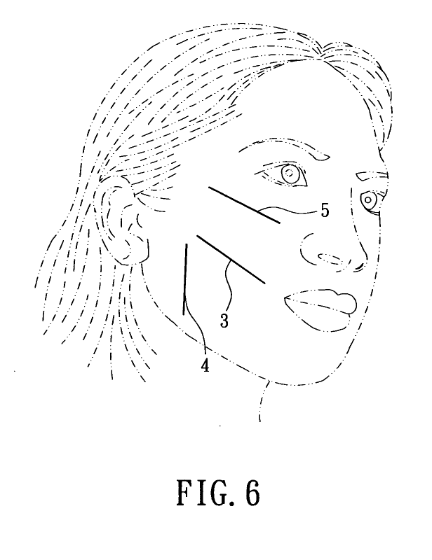

There are three preferable locations for attaching the pad. As shown in FIG. 6, the first location 3 is from cheekbone to lip corner. The effect of the stimulation is to activate and raise the cheek muscles. The second location 4 is from cheekbone to jawbone. The effect of the stimulation is to prevent from the formation of jowls and deformation of the face. The third location 5 is from temple to nostril. This place is a high sensitive area. The effect of the stimulation will obtain delighting enjoyment and exercise eyes. Moreover, same effect happens to massage these three locations, that is, increasing the blood circulation in surrounding areas so as to minimize the wrinkle and sagging of facial skin.

This disclosure provides exemplary embodiments of the present invention. The scope of this disclosure is not limited by these exemplary embodiments. Numerous variations, whether explicitly provided for by the specification or implied by the specification, such as variations in shape, structure, dimension, type of material or manufacturing process may be implemented by one of skill in the art in view of this disclosure.

1. An electrical impulse apparatus for facial massage, comprising:
a microprocessor;
an impulse generator controlled by the microprocessor to generate pulsed signals;
a switch connected to the microprocessor for inputting operation;
a pad with one surface attachable to facial skin; and
two electrodes mounted on the pad to electrically connect with the impulse generator;
thereby an electro-muscle stimulation is applied to facial tissue as a operation cycle including a first session of pulse generation with 100 Hz in frequency and 100 μs in width for continuous three minutes, a second session with 130 Hz and 80 μs for continuous three minutes, a third session with 100 Hz and 100 μs for continuous four minutes, a fourth session with 8 Hz and 20 μs for continuous two minutes, a fifth session with 100 Hz and 100 μs for continuous four minutes, a sixth session with 5 Hz and 30 μs for continuous two minutes, a seventh session with 130 Hz and 80 μs for continuous four minutes, a eighth session with 8 Hz and 20 μs for continuous two minutes, a ninth session with 130 Hz and 80 μs for continuous four minutes, and a tenth session with 5 Hz and 30 μs for continuous two minutes.

2. The apparatus of claim 1, wherein all sessions are continuous to obtain a totally thirty-minute operation cycle.

3. The apparatus of claim 1, further comprising a housing for installation of the microprocessor and the impulse generator inside, and the buttons on a surface thereof.

4. The apparatus of claim 3, wherein the pad is located directly on the housing.

5. The apparatus of claim 3, further comprising two conductive lines connected between the impulse generator and each of the electrodes, respectively.

6. The apparatus of claim 1, further comprising a light emitting device connected to the microprocessor for indicating an operation status.

7. The apparatus of claim 1, wherein the switch includes two buttons to increase and decrease the strength of the pulse generation, respectively.

8. The apparatus of claim 7, wherein the strength increasing button is a power-on switch at start.

9. The apparatus of claim 7, wherein the strength decreasing button is a power-off switch after sequent reduction.
